(19) United States
(12) Patent Application Publication
(10) Pub. No.: US 2025/0087360 A1
(43) Pub. Date: Mar. 13, 2025

(54) SYSTEM AND METHOD FOR CALCULATING WHOLE HEALTH INDEX

(71) Applicant: Elevance Health, Inc., Indianapolis, IN (US)

(72) Inventors: Winnie Chi, Indianapolis, IN (US); Kevin Bowman, Indianapolis, IN (US); Joseph M. Overhage, Indianapolis, IN (US); Shantanu Agrawal, Indianapolis, IN (US)

(73) Assignee: Elevance Health, Inc., Indianapolis, IN (US)

(21) Appl. No.: 18/505,540

(22) Filed: Nov. 9, 2023

Related U.S. Application Data
(60) Provisional application No. 63/581,113, filed on Sep. 7, 2023.

(51) Int. Cl. G16H 50/30; G16H 10/60; G16H 50/20

(52) U.S. Cl. CPC G16H 50/30; G16H 10/60; G16H 50/20

(57) ABSTRACT

A system includes an interface module that interfaces with disparate data sources for health data. The module monitors the data sources for updates, and extracts, transforms and loads health data. A data normalizing module normalizes the health data for a population. A domain selection and indicator selection module selects domains and indicators based on significance to health, validity of indicators, availability of the indicators, applicability of indicators for the population, and timeliness of the indicators. The module also selects a subset of indicators for computational time efficiency. A weight generation module generates weights for the domains and the subset of indicators based on a random sample of the health data. A whole health index calculation module calculates a weighted sum of the health data based on the weights to obtain a whole health index for the population.

### CROSS-REFERENCE TO RELATED APPLICATIONS

This application claims the benefit of U.S. Provisional Patent Application No. 63/581,113 filed Sep. 7, 2023, the entirety of which is incorporated herein by reference.

### TECHNICAL FIELD

The invention relates to systems and methods for healthcare systems, and is more particularly, but not by way of limitation, directed to technology for calculating health index.

### BACKGROUND

There is greater awareness across stakeholders in the healthcare ecosystem that there is a need to address social, physical, and clinical factors together to improve population health effectively. The first step in doing so is measuring whole-person health accurately, because it is not possible to improve what cannot be measured. However, very few measures are designed to measure health comprehensively, are tested to be valid and reliable, and are systematically computable in real-time.

Establishing a measure that wholistically assesses health is foundational to improving not only health, but also health equity. Considering that health and its determinants are multi-dimensional, an adequate measure of whole-person health (i.e., looking at the whole person—not just different components of the body) should be (a) inclusive of social, clinical, and physical factors, (b) valid and reliable, (c) systematically computable for a large majority of individuals in a population, (d) sensitive in distinguishing differences in health status among individuals, even those without disease, and (e) available promptly. However, few measures adequately meet all these criteria. Conventional measures and risk scores in population health management rely on diagnoses captured from administrative claims, such as the Charlson comorbidity index, the Elixhauser comorbidity index and the Centers for Medicare and Medicaid's Diagnostic Cost Group Hierarchical Condition Category. However, these measures do not differentiate health among those lacking access to health care. Other commonly used measures for comparing health across countries are based on mortality data, such as life expectancy, or disability-adjusted life years (DALY). However, those measures are typically not computable at the individual level. Self-reported measures of health are valuable indicators of individuals' perception of their health. However, they are costly to measure and may be subject to reporting bias. Another group of indices focus exclusively on social factors, such as the University of Wisconsin's Area Deprivation Index (ADI) and the Centers of Disease Control and Prevention's Social Vulnerability Index (SVI).

### SUMMARY

Accordingly, there is a need for tools, systems and methods for calculating whole health index. Some embodiments calculate a whole health index (WHI) as a composite score that measures an individual's health by incorporating geographic level health factors with individual health factors such as social needs, clinical quality measures and diagnoses. Some embodiments compute the WHI based on the Institute of Medicine's Vital Sign framework. Some embodiments compute the WHI for millions of members using enrollment and claims data combined with publicly available data as inputs to the index. In some embodiments, WHI has three domains representing physical, clinical, and social factors affecting health: (1) global health, focusing on the presence of conditions and diseases; (2) clinical quality, derived from the healthcare effectiveness data and information set (HEDIS) and other validated quality measures; and (3) social drivers, capturing neighborhood factors, social needs, and healthcare affordability. The WHI was assessed for criterion validity, convergent validity, discriminant validity, and reliability. Analyses demonstrated that the WHI is a valid measure of whole-person health at both individual level and several levels of geography, including at census tract-, 5-digit ZIP Code-, and county-levels.

In one embodiment, as a composite score of these three domain scores, the WHI has a range from 0 (worst health) to 100 (best health). Out of millions of members, the WHI score ranged from 9.17 to 90.75, with an average of 53.08, a median of 53.23 (interquartile range (IQR): 43.34, 62.95), and an approximately bell-shaped distribution.

The WHI can be used as a tool to measure whole-person health, to inform population health program planning and to foster cross-care team collaboration. Improving population health requires partnership and collaboration across multiple stakeholders. The WHI scoring is a transparent way to measure health.

One or more embodiments of the invention are directed to an improved method and system for calculating whole health index (WHI). The method may be performed at a server having one or more processors, memory, one or more displays, and one or more programs stored in the memory and configured for execution by the one or more processors, the one or more programs including instructions for performing the steps described herein. The method includes interfacing with a plurality of disparate data sources, via a data cloud. The plurality of disparate data sources includes (i) at least one data source for managing health plan enrollments and claims data, and (ii) at least one data source storing public health data. The method also includes receiving and normalizing health data from the plurality of disparate data sources, the health data including (i) health plan enrollments and claims data, and (ii) public health data, for a population. The method also includes selecting a plurality of domains and a plurality of indicators based on (i) significance to health, (ii) validity of the indicators, (iii) availability of the indicators at large scales, (iv) applicability of indicators to the broader population, and (v) timeliness of the indicators. The method may also include selecting a subset of indicators for the plurality of domains using a random sample of the health data to assess the indicators for computational time efficiency. The method may also include removing indicators that (i) amount to incomplete data capture using the plurality of disparate data sources or (ii) applicable only to a subset of the population. The method also includes generating weights for each of the plurality of domains and the subset of indicators based on the random sample of the health data. The method also includes calculating a weighted sum of the health data based on the weights to obtain a whole health index for the population.

In some embodiments, generating the weights includes calculating the whole health index under a plurality of weighting schemes to determine weighting schemes across the plurality of domains and subdomains, and selecting a final weighting scheme based on an option that yields validation results in accordance with a predetermined criterion.

In some embodiments, selecting the final weighting scheme includes examining the criterion validity of the whole health index by analyzing Spearmen correlation between average whole health index at a county level and public health indicators including length of life (life expectancy) and quality of life (e.g., self-reported healthy days measures, including CDC healthy days, frequent mental distress, frequent physical distress, mental health day, and/or physically healthy days).

In some embodiments, selecting the final weighting scheme includes assessing if the whole health index reflects known differences in health across different populations, based on age groups, sex, race/ethnicities, rural/urban status, and/or insurance types, and selecting the final weighting scheme by determining if a scheme yields a predetermined level of performance in terms of criterion validity and discriminant validity.

In some embodiments, the method further includes, in accordance with a determination that individuals in the whole population have missing scores in global health or clinical quality domains, imputing scores based on median domain score of other individuals in the same age band and sex living in the same state, respectively.

In some embodiments, the method further includes, in accordance with a determination that individuals in the whole population have missing social driver scores, imputing social driver score based on median value among other individuals with the same insurance types and living in the same state.

In some embodiments, generating the weights includes validating the whole health index on a predetermined portion of the health data, including analyzing Spearman correlation between average whole health index at county level and predetermined health indicators at county-level, based on health indicators comprising length of life and quality of life.

In some embodiments, generating the weights includes assessing validity of whole health index, including estimating construct validity of composite of the whole health index, computing correlations (e.g., Pearson, Spearman, intra-class) between three domains, conditioning these correlations on number of conditions present.

In some embodiments, generating the weights includes assessing discriminant validity by determining if the whole health index scores reflect an expected impact of clinical conditions, including assessing if individuals with multiple conditions have lower whole health index on average compared to individuals without multiple conditions, and if individuals with more severe health conditions have lower whole health index compared to those with less severe health conditions.

In some embodiments, generating the weights includes evaluating reliability of the whole health index at varying levels of geography by assessing stability of the whole health index scores.

In some embodiments, evaluating reliability includes computing split-half reliability of the whole health index scores at county and 5-digit ZIP Code levels by performing one or more of the following steps: splitting individuals within a geographical level into two groups using random sampling; computing area-level whole health index scores in both samples; and computing Pearson, Spearman, and intra-class correlations for the whole health index scores across two samples.

In some embodiments, evaluating reliability includes assessing precision of whole health index scores across various levels of geography, including computing within geographic unit variance to between geographic unit variance (WGVBGV) of the whole health index scores at census tract, 5-digit ZIP Code, and county level. WGVBGV, using the terminology of a signal-to-noise ratio, is the ratio of signal variance to the sum of the signal and noise variances (total variance in the measure). The WGVBGV statistic, ranging from 0 to 1, summarizes the proportion of the total variation in the whole health index scores at the area level due to differences between areas (considered as the signal) in relation to individual-level variation within each area (considered noise for the purposes of this test). If WGVBGV is equal to 1, variation in whole health index scores is due to differences in quality observed at the geographic level. If WGVBGV is close to zero, whole health index scores are not driven by differences in health but rather by random variation and will therefore not be useful to compare health across areas.

In some embodiments, the method further includes subdividing the health data corresponding to social drivers domain into data for six subdomains for (1) financial strain, (2) healthcare affordability, (3) food insecurity, (4) transportation barriers, (5) housing insecurity, and (6) minority status and language. The method may further include generating weights for each of the subdomains, which in turn may include calculating subdomain scores by combining individual and area-level data with equal weights, and in accordance with a determination that individuals did not have individual-level social driver data, using area-level data for the subdomain scores. The method may further include calculating the weighted domain for the social drivers domain by summing percentiles of each subdomain multiplied by a weighting factor.

In some embodiments, the method further includes subdividing the health data corresponding to clinical quality domain into data for six subdomains for (1) access to care, prevention, and screening, (2) acute care and care coordination, (3) overuse, appropriateness, and safety, (4) cardiovascular conditions, diabetes, oncology, and respiratory conditions, (5) behavioral health, and (6) women's health. The method may further include generating weights for each of the subdomains. Generating weights for the subdomains may include assigning higher weights to subdomains with more measures and more direct impact on wellbeing than other subdomains. Generating weights for the subdomains may include identifying measures within each subdomain as either a process or an outcome measures, and using a 1:3 process-to-outcome ratio to weight outcome measures more heavily. Generating weights for the subdomains may include calculating subdomain scores by combining individual and area-level data with equal weights. Generating weights for the subdomains may include scoring individuals only for measures they are qualified for. The method may further include calculating the weighted domain for the clinical quality domain by summing percentiles of each subdomain multiplied by a weighting factor.

In some embodiments, the method further includes providing each domain score to a plurality of computing resources corresponding to care teams to identify potential needs beyond their clinical program offering, and to provide additional care solutions, such as meal delivery services, transportation support, or hearing aid consultation, to improve whole health for the population.

In some embodiments, the method further includes using the whole health index to direct members of the population to appropriate solutions for their specific health and social needs.

In some embodiments, a computer system has one or more processors, memory, and a display. The one or more programs include instructions for performing any of the methods described herein.

In some embodiments, a non-transitory computer readable storage medium stores one or more programs configured for execution by a computer system having one or more processors, memory, and a display. The one or more programs include instructions for performing any of the methods described herein.

### BRIEF DESCRIPTION OF THE DRAWINGS

FIG. 1 is a schematic diagram of an example system for calculating whole health index, according to some embodiments;

FIG. 2 is a system diagram of an example whole health index calculation server 102, according to some embodiments;

FIG. 3 is a schematic diagram of different domains for calculating whole health index, according to some embodiments;

FIG. 4 is a graph plot for example distribution of the whole health index among individuals in a population, according to some embodiments;

FIG. 5 is a map showing the average whole health index by county in the US, according to some embodiments;

FIG. 6 is a bar chart 600 showing example correlation between the average whole health index and other health indicators at county level, according to some embodiments;

FIG. 7 shows a table of example indicators for the social driver domain, according to some embodiments;

FIGS. 8A-8C show a table of example indicators for measure weights in the clinical quality domain, according to some embodiments;

FIG. 9A shows a table for example WGVBGV validation results, according to some embodiments;

FIG. 9B shows a table for example split-half test correlation coefficients, according to some embodiments;

FIG. 10 is a bar chart showing average whole health index by population segments and clinical acuity, according to some embodiments;

FIG. 11 shows a flowchart of an example method for calculating whole health index, according to some embodiments; and

FIG. 12 is a block diagram of an example system for whole health index calculation, according to some embodiments.

### DETAILED DESCRIPTION OF THE PREFERRED EMBODIMENTS

The following descriptions of embodiments of the invention are exemplary, rather than limiting, and many variations and modifications are within the scope and spirit of the invention. Although numerous specific details are set forth in order to provide a thorough understanding of the present invention, it will be apparent to one of ordinary skill in the art, that embodiments of the invention may be practiced without these specific details. In other instances, well-known features have not been described in detail in order to avoid unnecessarily obscuring the present invention.

One or more embodiments of the invention are directed to an improved method and system for calculating whole health index.

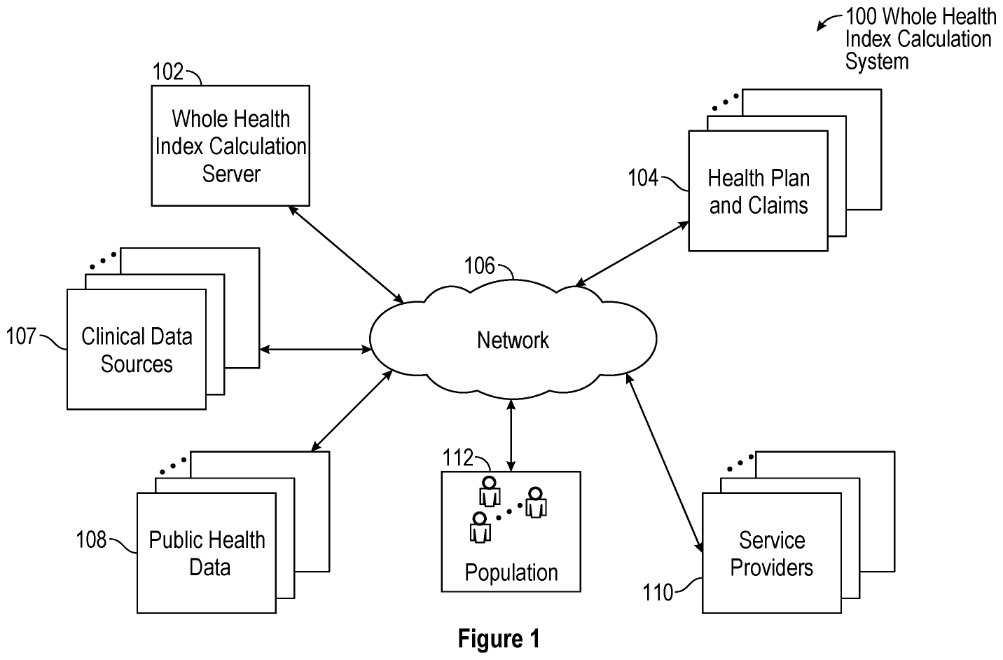

FIG. 1 is a schematic diagram of an example system 100 for calculating whole health index, according to some embodiments. The system 100 may include a whole health index calculation server 102 coupled to a plurality of data sources, including one or more health plan and claims sources 104, one or more clinical data sources 107, and one or more public health data sources 108. The whole health index calculation server 102 may be coupled to devices (e.g., client devices, such as mobile devices) corresponding to a population 112 (e.g., individuals in an area, individuals under a health care plan) whose whole health index is calculated. The whole health index calculation server 102 may also be coupled to computing resources corresponding to one or more service providers 110 (e.g., care teams to identify potential needs beyond their clinical program offering, and care solutions, such as meal delivery services, transportation support, or hearing aid consultants, to improve whole health for the population).

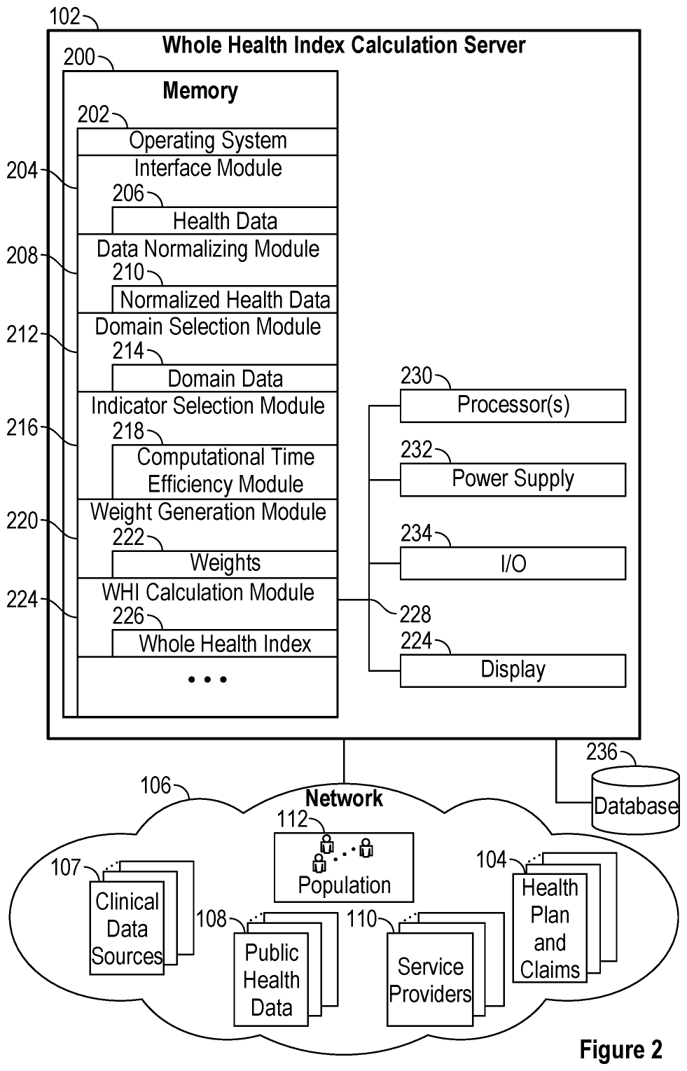

FIG. 2 is a system diagram of an example whole health index calculation server 102, according to some embodiments. The whole health index calculation server 102 typically includes one or more processor(s) 230, a memory 200, a power supply 232, an input/output (I/O) subsystem 234, and a communication bus 228 for interconnecting these components. Processor(s) 230) execute modules, programs and/or instructions stored in memory 200 and thereby perform processing operations, including the methods described herein according to some embodiments. In some embodiments, the information security risk manager 102 also includes a display 244 for displaying visualizations (e.g., risk scores, probabilities). In some embodiments, the whole health index calculation server 102 generates displays or visualizations, and transmits the visualization (e.g., as a visual specification) to a client device for display. Some embodiments of the whole health index calculation server 102 include touch, selection, or other I/O mechanisms coupled to the whole health index calculation server 102 via the I/O subsystem 234, to process input from users that select (or deselect) visual elements of a displayed visualization. Some aspects of the information security risk manager 102 (e.g., the modules in the memory 200) are implemented in one or more client devices, according to some embodiments. In some embodiments, the client device (or software therein) processes user input and transmits a signal to the whole health index calculation server 102 for processing.

In some embodiments, the memory 200 stores one or more programs (e.g., sets of instructions), and/or data structures, collectively referred to as “modules” herein. In some embodiments, the memory 200, or the non-transitory computer readable storage medium of the memory 200, stores the following programs, modules, and data structures, or a subset or superset thereof:
an operating system 202;
an interface module 204 that interfaces with a plurality of data sources (e.g., the clinical data 107, the public health data 108, the health plan and claims 104) to monitor updates for and/or obtain health data 206 from the data sources;
a data normalizing module 208 normalizes health data 206 obtained from the data sources 206;
a domain selection module 212 selects domain data 214 from the normalized health data 210;
an indicator selection module 216 selects indicators of whole health. Some embodiments use a computational time efficiency module 218;
a weight generation module 2210 generates weights 222 for computing whole health index; and
a whole health index calculation module 224 for calculating whole health index 226 based on the weights 222.

The above identified modules (e.g., data structures, and/or programs including sets of instructions) need not be implemented as separate software programs, procedures, or modules, and thus various subsets of these modules may be combined or otherwise re-arranged in various embodiments. In some embodiments, memory 202 stores a subset of the modules identified above. In some embodiments, a database 236 (e.g., a local database and/or a remote database) stores one or more modules identified above and data associated with the modules. Furthermore, the memory 200 may store additional modules not described above. In some embodiments, the modules stored in memory 200, or a non-transitory computer readable storage medium of memory 200, provide instructions for implementing respective operations in the methods described below. In some embodiments, some or all of these modules may be implemented with specialized hardware circuits that subsume part or all of the module functionality. One or more of the above identified elements may be executed by the one or more of processor(s) 230.

I/O subsystem 234 communicatively couples the whole health index calculation server 102 to one or more devices such as the client devices corresponding to the population 112, the health plan and claims data sources 104, the clinical data sources 107, and/or the public health data 108, via a local and/or wide area communications network 106 (e.g., the Internet) via a wired and/or wireless connection. In some embodiments, the client devices corresponding to the population 112, the health plan and claims data sources 104, the clinical data sources 107, and/or the public health data 108 push relevant information to the whole health index calculation server 102. In some embodiments, the whole health index calculation server 102 pulls relevant information from the client devices corresponding to the population 112, the health plan and claims data sources 104, the clinical data sources 107, and/or the public health data 108.

Communication bus 228 optionally includes circuitry (sometimes called a chipset) that interconnects and controls communications between system components.

Some embodiments combine area-level and individual-level social and clinical risk factors, representing key determinants of health. Some embodiments generate a single composite score that measures an individual's health among the general population that is referred to as the whole health index. Some embodiments use the National Academy of Medicine's Vital Signs framework to inform the domain and indicator selection for the whole health index. The choice for domains and indicators may be based on multiple considerations including (a) significance to health, (b) validity, (c) availability at large scale, (d) applicability to the broader population, and (e) timeliness. Some embodiments compute a numeric measure of health that can be used to track health for a same individual over time and to compare health across populations to inform meaningful actions to improve health and health equity.

### Example Methods for Computing Whole Health Index

### Example Data Sources

Some embodiments combine health enrollment, claims and clinical data with publicly available data to compute the whole health index for health plan members (e.g., health plan members who had at least one day of medical plan eligibility during a period). The data sources 107, 108 and 104 may represent disparate data sources, the data may be formatted differently and/or may include anonymized health data. The data normalizing module 208 normalizes such data across the disparate data sources. The individuals that correspond to the health data may be covered by multiple insurance types, including commercial plans, Medicaid, Medicare Advantage, and other supplemental health care plans. Global health and clinical quality measures may be drawn from enrollment, claims and clinical data. A social driver domain may include individual-level and area-level measures for assessing social need, including z-codes, LOINC codes, census-tract-level measures (e.g., measures from the 2020 5-year American Community Survey and the Environmental Protection Agency), and county-level measures (e.g., measures from the 2021 County Health Ranking data). Healthcare affordability may be calculated as total out-of-pocket spending (the sum of copay, coinsurance, and deductible amounts during the measurement period as recorded on claims data) divided by the median household incomes at the census-tract level from the censes-tract-level measures (e.g., 2020 5-year American Community Survey). Some embodiments use a dataset for analysis of the managed care organization's membership data for the purposes of health plan treatment, planning, and operations. The dataset may not have individual patient identifiers, and may comply with provisions of the Health Insurance Portability and Accountability Act. Such data may be stored as part of the health data 206.

### Example Domains

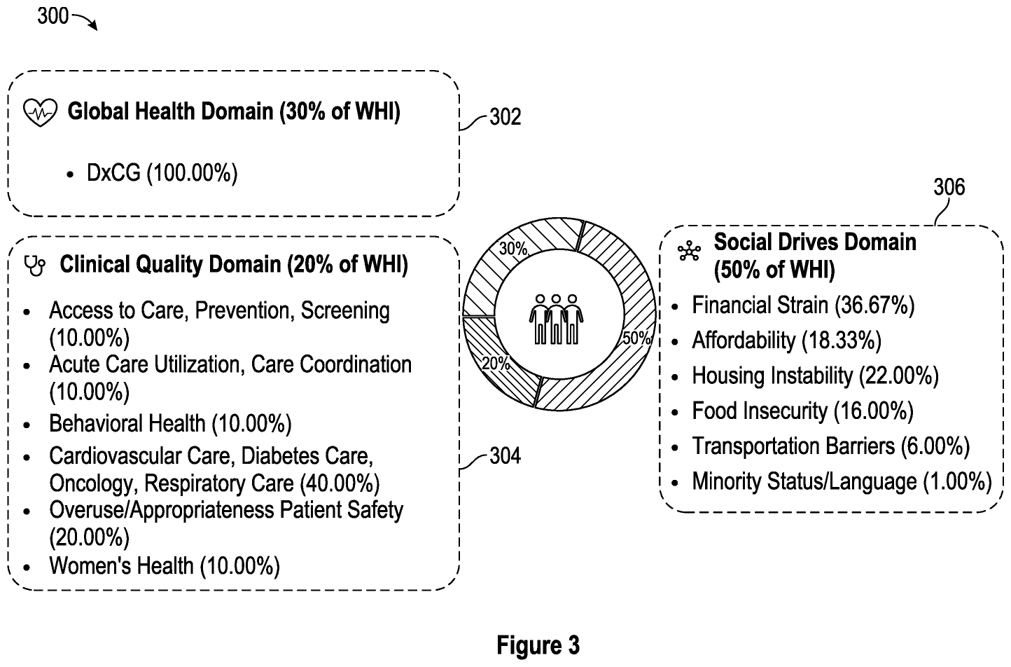

FIG. 3 is a schematic diagram of different domains 300 for calculating whole health index, according to some embodiments. Example weights for the sub-domains (contributions of the factors to WHI) are shown within parentheses. A global health domain 302 (e.g., 30% of WHI) may include age, sex and a comorbidity score, such as Elixhauser comorbidity index (e.g., 100%). A clinical quality domain 304 (e.g., 20% of WHI) may include (i) access to care, prevention, screening (e.g., 10%), (ii) acute care utilization, care coordination (e.g., 10%), (iii) behavioral health (e.g., 10%), (iv) cardiovascular care, diabetes care, oncology, respiratory care (e.g., 40%), (v) overuse or appropriateness, patient safety (e.g., 20%) and/or women's health (e.g., 10%). Social drivers domain 306 (e.g., 50% of WHI) may include (i) financial strain (e.g., 36.67%), affordability (e.g., 18.33%), housing instability (e.g., 22%), food insecurity (e.g., 16%), transportation barriers (e.g., 6%), and minority status or language (e.g., 1%).

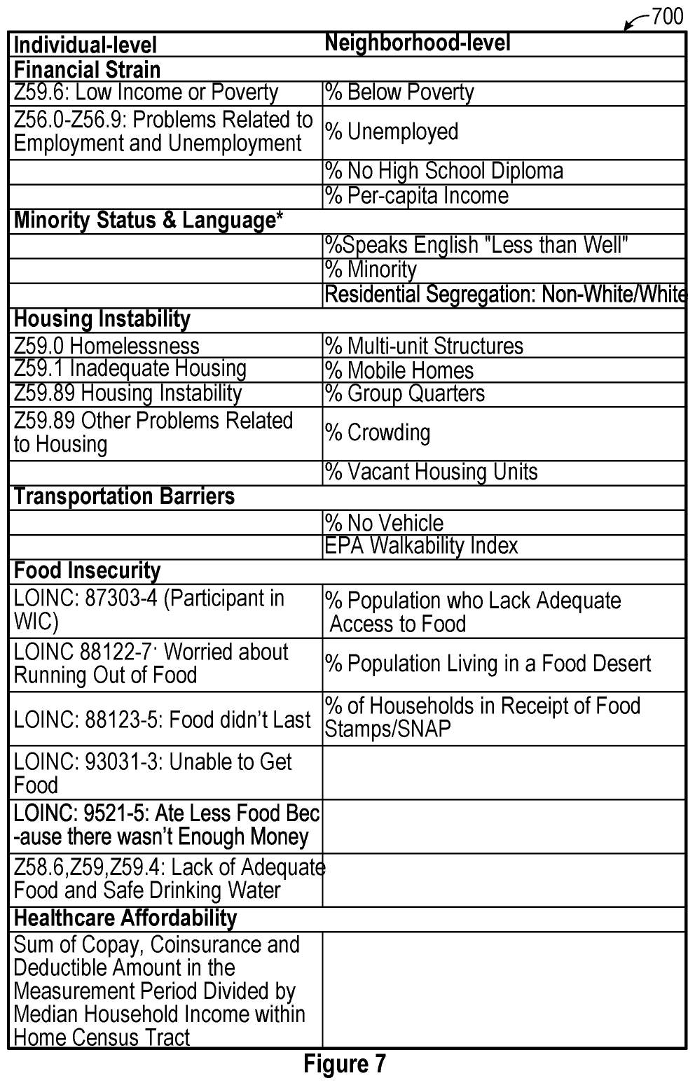

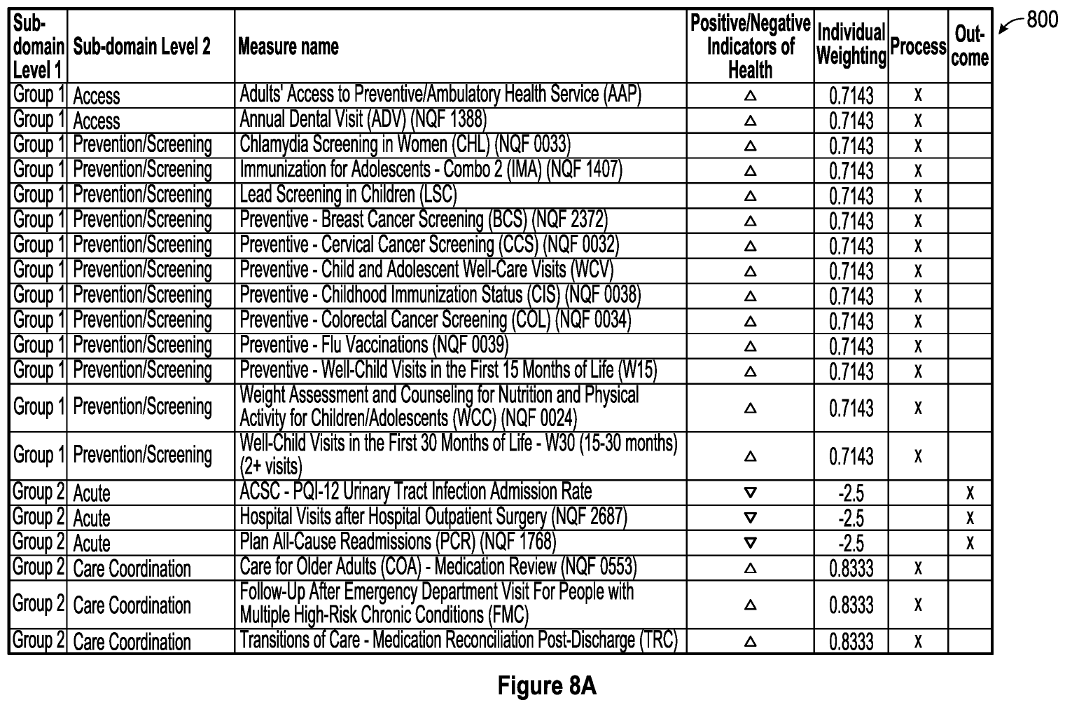

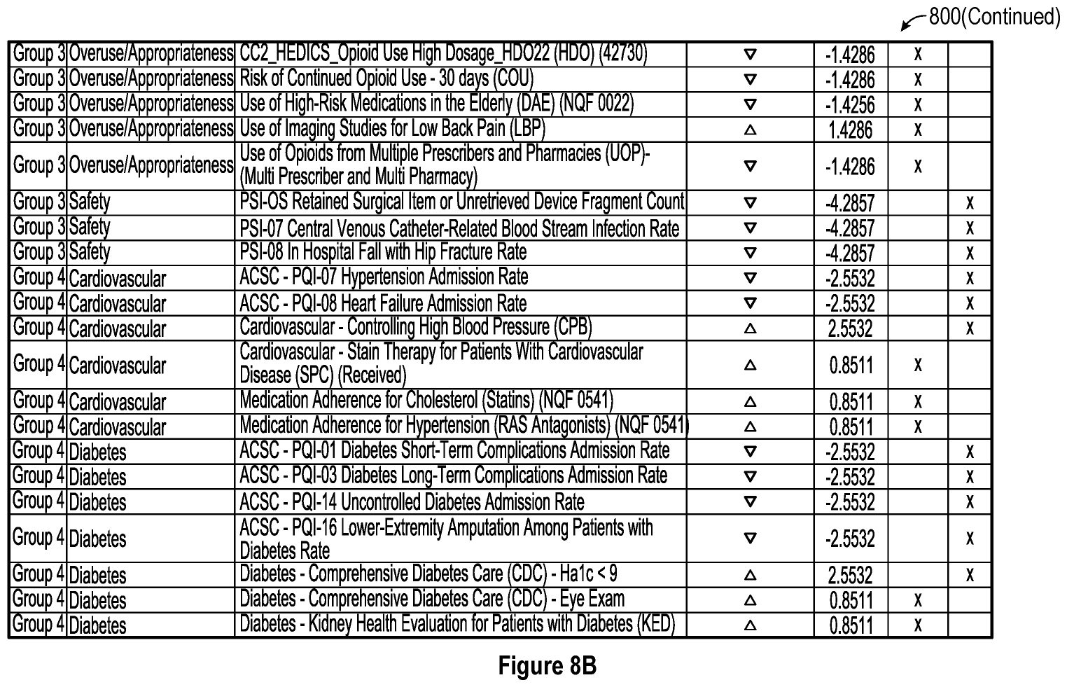

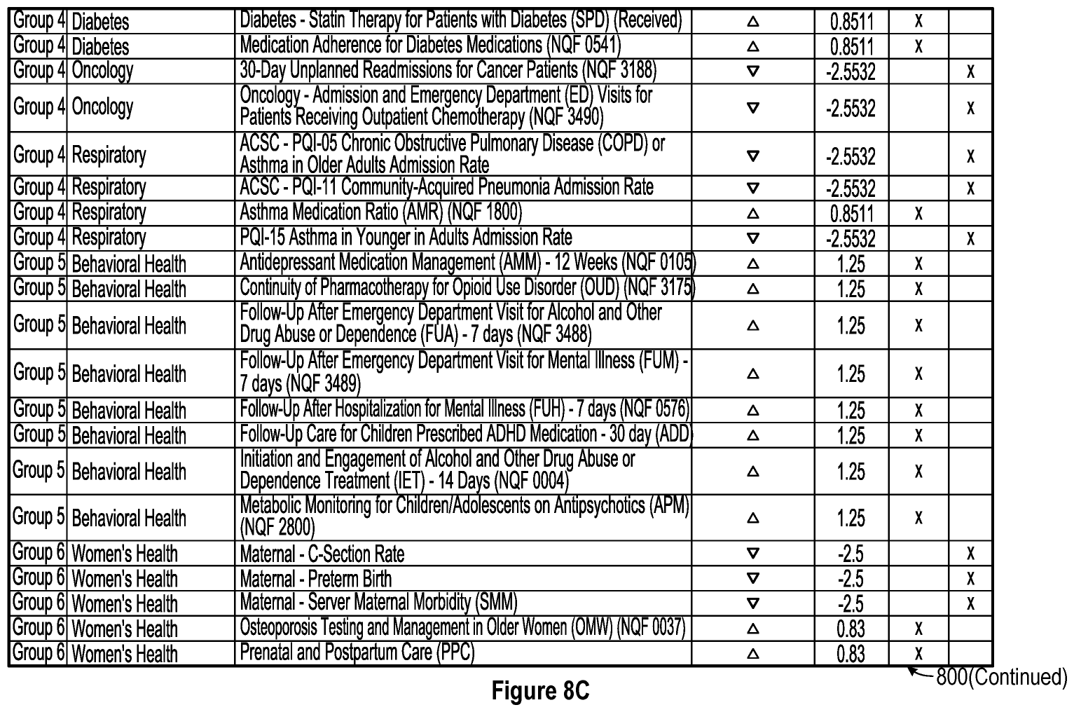

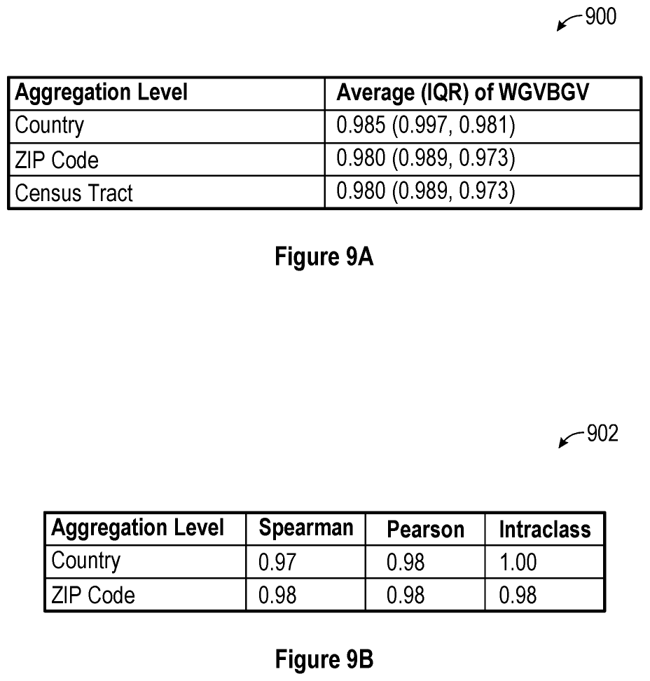

In some embodiments, the whole health index is a composite measure in which three domains (global health 302, clinical quality 304, and social drivers 306) capture different aspects of whole-person health. Each domain comprises an array of indicators. FIG. 7 shows a table of example indicators 700 for the social driver domain 306, according to some embodiments. FIGS. 8A-8C show a table of example indicators 800 for measure weights in the clinical quality domain 304, according to some embodiments. In some embodiments, the three domains of the whole health index are weighted as follows: social drivers, 50%; global health, 30%; clinical quality, 20% as informed by the National Academy of Medicine's Vital Signs framework. In some embodiments, the whole health index score is calculated as the weighted sum of the three-domain scores. The resulting whole health index was assessed for criterion validity, convergent validity, discriminant validity, and reliability at individual level and at aggregated level. Assessments demonstrated that the whole health index is a valid measure of whole-person health at both individual level and several levels of geography, including at census tract-, 5-digit ZIP Code-, and county-levels (e.g., FIGS. 9A and 9B, described below).

In some embodiments, the global health domain measures disease burden an individual experienced during the measurement period. Some embodiments use age, sex, and comorbidity scores as a summary measure. The age, sex, and comorbidity scores predict total healthcare costs, including plan paid and patient paid amount, based upon demographic and clinical information reported in a 12-month period. Higher scores indicate higher predicted total healthcare cost. The total healthcare cost represents overall healthcare utilization more comprehensively, thus is a better proxy for disease burden, as opposed to plan paid cost. Some embodiments replace the age, sex, and comorbidity scores with another disease burden measure in public domains. In some embodiments, the global health domain score is calculated as the percentile ranking of health plan members according to the age, sex, and comorbidity scores distribution in a calendar year (a baseline year for benchmarking), with higher percentiles indicating lower age, sex, and comorbidity scores and better health.

In some embodiments, the social driver domain is constructed as the weighted summation of six subdomains: (1) financial strain, (2) healthcare affordability, (3) food insecurity, (4) transportation barriers, (5) housing insecurity, and (6) minority status and language. The subdomain scores may be calculated by combining individual and area-level data with equal weights (50% and 50%). In the case where individuals did not have individual-level social driver data, the subdomain scores may use area-level data alone. The social driver score may be calculated by summing the percentiles of each subdomain multiplied by a weighting factor (e.g., FIG. 3). Table shown in FIG. 7 lists example data elements included in the social driver domain, according to some embodiments.

In some embodiments, the clinical quality domain is based on 63 clinical quality factors grouped into six subdomains: (1) access to care, prevention, and screening, (2) acute care and care coordination, (3) overuse, appropriateness, and safety, (4) cardiovascular conditions, diabetes, oncology, and respiratory conditions, (5) behavioral health, and (6) women's health. In some embodiments, subdomains are weighted such that those with more measures and more direct impact on wellbeing are given higher weights. In some embodiments, measures within each subdomain are identified as ‘process’ or ‘outcome’ measures, and a 1:3 process-to-outcome ratio is used to weight outcome measures more heavily. Table shown in FIGS. 8A-8C lists the weighting for each quality measure, according to some embodiments. Individuals may be only scored for the measures for which they are qualified. If an individual qualified for the denominator of a given measure and met the criteria for the numerator, they may be given a weight specific to that measure. For example, if an individual is qualified for colorectal cancer screening and is compliant with the screening, then an individual may receive positive points of the measure weight (0.7143); however if not compliant, an individual may receive negative points of the measure weight (−0.7143).

### Example Whole Health Index Scoring Method

In some embodiments, the National Academy of Medicine's Vital Signs framework is used for calculating the whole health index for domain selection (by the domain selection module 212) and indicator selection (by the indicator selection module 216) as described below. An expert panel consisting of clinicians, subject matters experts in clinical quality and social drivers, and population health researchers, may also help inform the selections. The choice for domains and indicators may be based on one or more factors: (a) significance to health, (b) validity of the indicators, (c) availability of the indicators at large scales, (d) applicability of indicators to the broader population, and (e) timeliness of the indicators.

After selecting domains and a potential list of indicators, some embodiments of the computational time efficiency module 218 use a random sample (e.g., randomly selected 10% of a training data) to assess the indicators for computational time efficiency. Some embodiments of the weight generation module 220 also use the 10% random sample to develop a weighting scheme and validate the methodology before applying the scoring methodology to individuals in a study population.

Some embodiments of the WHI calculation module 224 calculate the whole health index under different weighting schemes (e.g., 100 different schemes) to determine weighting schemes (corresponding to different weights 222) across domains and subdomains. A final weighting scheme may be selected by the weight generation module 220) and/or the WHO calculation module 224, based on the option that yields the strongest validation results in terms of criterion validity. Criterion validity refers to how well a measure is correlated with the gold standard of what the measure is intended to measure. Strongest results are results that are the best among all the different weighting scenarios tested. Some embodiments examine the criterion validity of the whole health index by analyzing the Spearmen correlation between the average whole health index at the county level and several known health indicators. Some embodiments use a plurality of health indicators, including one or more indicators selected from the group consisting of: length of life (life expectancy) and quality of life (e.g., self-reported healthy days measures, such as CDC healthy days, frequent mental distress, frequent physical distress, mental health day, physical healthy day, etc.). Some embodiments use data compiled from the County Health Rankings and the Behavioral Risk Factor Surveillance System 2018. Some embodiments assess if the whole health index reflects known differences in health across different populations, based on age groups, sex, races/ethnicities, rural/urban status, and insurance types. The final weighting scheme may yield the best performance in terms of criterion validity and discriminant validity.

Experiments showed that over 90% of individuals had scores available for all three domains; individuals rarely had missing scores across all three domains. When individuals had missing scores in the global health or clinical quality domains, the scores were imputed based on the median domain score of individuals in the same age band and sex living in the same state, respectively. When individuals had missing social driver scores (primarily due to missing address information), the social driver scores were imputed based on the median value among individuals with the same insurance types and living in the same state.

### Example Validation Process

In some embodiments, the validation is conducted on a 10% sample, consisting of approximately 4 million members. Criterion validity may be examined to determine the whole health index weighting methodology by analyzing the Spearman correlation between the average whole health index at the county level and several known health indicators at county level from the County Health Ranking 2022 data. A plurality of health indicators, including length of life (life expectancy) and quality of life (including CDC healthy days, frequent mental distress, frequent physical distress, mental health days, and physical healthy days) may be used.

The validity of whole health index may be assessed. To estimate validity of the composite, correlations (e.g., Pearson, Spearman, and intra-class) between different domains may be computed. These correlations may be conditioned on the number of conditions present, given that clinical quality scores are correlated with individuals healthcare needs. Given that the whole health index is a formative composite measure of health, all three domains may exhibit weak, positive Pearson correlations to each other. To assess discriminant validity, some embodiments assess whether the whole health index scores reflected the expected impact of clinical conditions. For example, some embodiments assess if individuals with multiple conditions have lower whole health index on average compared to individuals without multiple conditions, and if individuals with more severe health conditions have lower whole health index compared to those with less severe health conditions.

Some embodiments evaluate the reliability of the whole health index at varying levels of geography by assessing stability of the whole health index scores. Some embodiments compute split-half reliability of the whole health index scores at county and 5-digit ZIP Code levels using one or more of the following steps: (i) splitting the individuals within a geographical level into two groups using random sampling, (ii) computing area-level whole health index scores in both samples, and (iii) computing Pearson, Spearman, and intra-class correlations for the whole health index scores across two samples. Additionally, to assess the precision of whole health index scores across various levels of geography, some embodiments compute within geographic unit variance to between geographic unit variance (WGVBGV) of the whole health index scores at census tract, 5-digit ZIP Code, and county level. WGVBGV, using the terminology of a signal-to-noise ratio, is the ratio of signal variance to the sum of the signal and noise variances (total variance in the measure). The WGVBGV statistic, ranging from 0 to 1, summarizes the proportion of the total variation in the whole health index scores at the area level due to differences between areas (considered as the signal) in relation to individual-level variation within each area (considered noise for the purposes of this test). If WGVBGV is equal to 1, all variation in whole health index scores is due to differences in quality observed at the geographic level. If WGVBGV is close to zero, differences in health do not drive whole health index scores but rather due to random variation and will therefore not be useful to compare health across areas.

### Example Whole Health Index Validation Results

FIG. 9A shows a table for example WGVBGV validation results 900, according to some embodiments. For a measure to be valid, it needs to be reliable. The WGVBGV results show that whole health index is a reliable measure at not only the county level, but also at the 5-digit ZIP Code and census tract levels. The average (IQR) of WGVBGV at county, ZIP Code, and census tract levels were 0.985 (0.997, 0.981), 0.980 (0.989, 0.973), and 0.980 (0.989, 0.973), respectively, which were all higher than 0.7, a commonly used threshold for measure reliability.

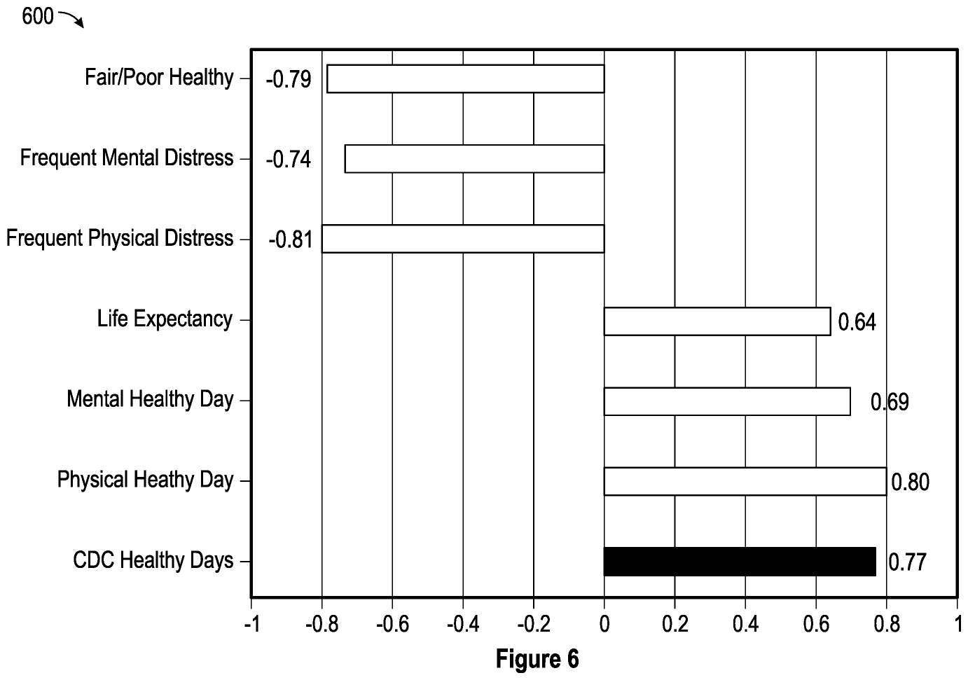

FIG. 9B shows a table for example split-half test correlation coefficients 902, according to some embodiments. Based on the split-half test, the whole health index is reliable at the county level with a Spearman correlation of 0.97 and a Pearson correlation of 0.98, along with ICC=1.00, as well as at the ZIP Code level. The Index has a moderate-to-strong correlation (0.64-0.81) with other known health indicators at the same geographic level. The Index has a positive correlation with the good health indicators (such as longer life expectancy, more CDC healthy days) and negative correlation with poor health indicators (such as self-reported fair or poor health, frequent mental and physical distress) (FIG. 6). These results demonstrate that the whole health index has a desired criterion validity.

All three domains were positively and weakly correlated with each other and the Pearson correlation at the individual level ranged from 0.05-0.14 (p<0.001), providing evidence of construct validity given that the whole health index is a formative composite measure. For discriminant validity, the whole health index could differentiate differences in health across different populations. Relatively socially vulnerable population subgroups, including older adults, females, the dual eligible population, rural residents, and Blacks, Hispanics, or Native Americans have lower whole health index scores. Individuals who are the top 10% of the healthcare utilizer within their residence states also have lower whole health index scores. Examining the ability of the whole health index to discriminate members' clinical acuity, individuals with more conditions have lower whole health index compared to individuals with fewer conditions (p<0.0001). Individuals with COPD, lung cancer, or stroke also tend to have lower whole health index compared to others without such conditions (p<0.0001). Individuals with common yet manageable conditions such as diabetes, dyslipidemia, and depression have higher whole health index compared to individuals with the more serious conditions of COPD, lung cancer, stroke, chronic kidney disease and heart failure (p<0.0001). These findings supported discriminant validity of the whole health index.

### Example Results

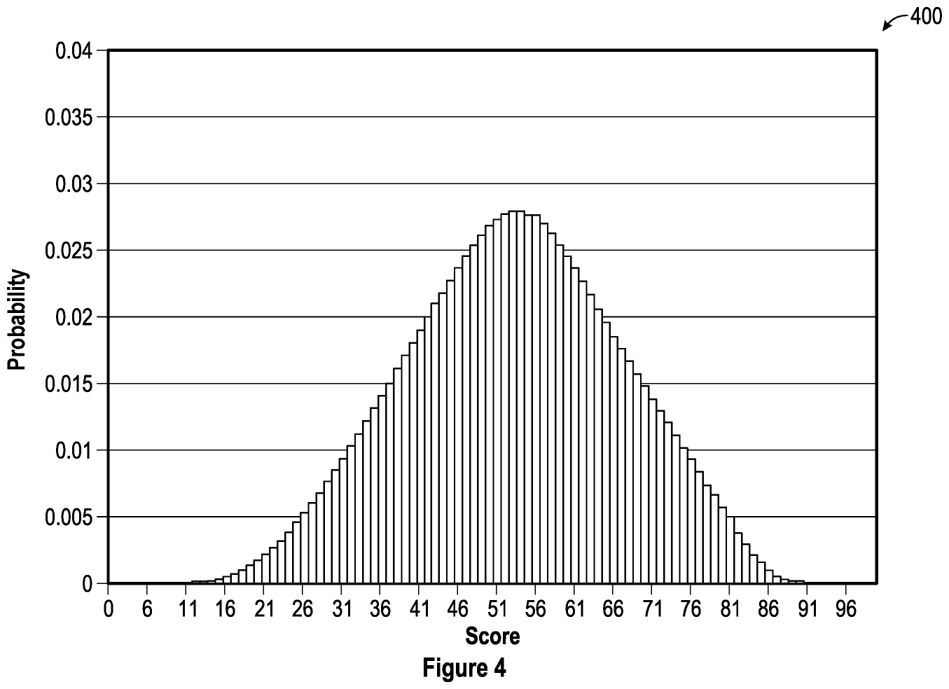

FIG. 4 is a graph plot for example distribution 400 of the whole health index among individuals in a population, according to some embodiments. In some embodiments, the whole health index has a theoretical range from 0 (worst health) to 100 (best health). In experiments, a sample of nearly 45 million Elevance Health members, the whole health index ranged from 9.17 to 90.75 with an average of 53.08 and a median of 53.23 (IQR: 43.34, 62.95) with a standard deviation of 13.86 and an approximately bell-shaped distribution (as shown in FIG. 4).

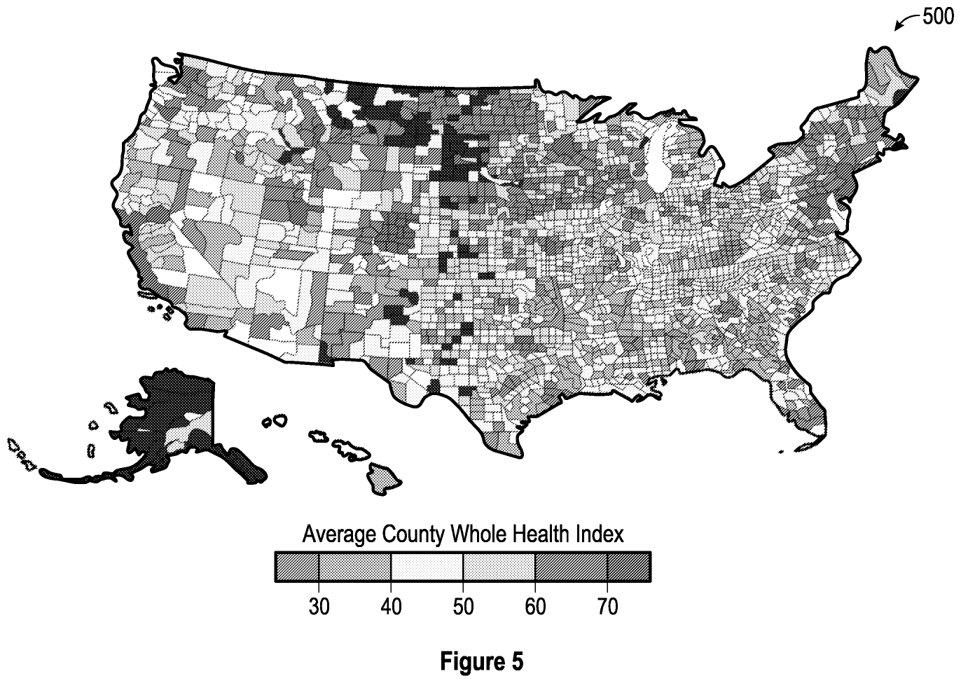

FIG. 5 is a map 500 showing the average whole health index by county in the US, according to some embodiments. Lower scores exist among states in the South region compared to states in the New England region. The whole health index can help identify counties associated with poor health outcomes. For example, Marion County, where Indianapolis is located, has a lower than average whole health index score, whereas Boone County and Hamilton County, located north of and adjacent to Marion County, have higher than the average whole health index scores. These findings are consistent with results from the 2020 County Health Ranking report. FIG. 6 is a bar chart 600 showing example correlation between the average whole health index and other health indicators at county level, according to some embodiments. The whole health index has a moderate-to-strong correlation (0.64-0.81) with other known health indicators at county level.

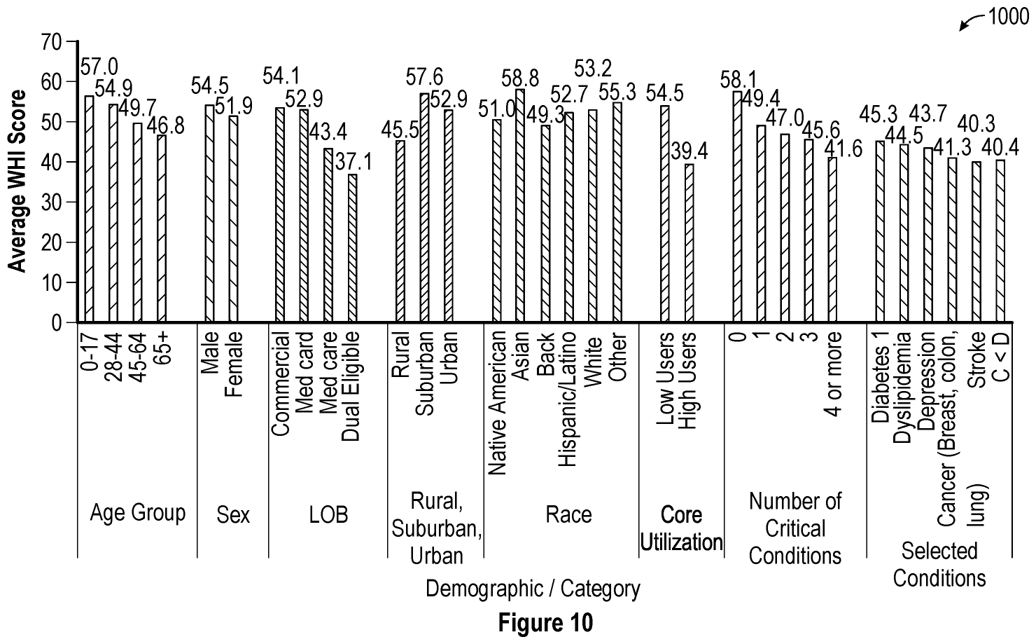

FIG. 10 is a bar chart 1000 showing average whole health index by population segments and clinical acuity, according to some embodiments. Relatively socially vulnerable population subgroups, including older adults, females, the dual eligible population, rural residents, and Blacks, Hispanics, or Native Americans have lower whole health index scores. Individuals with more conditions have lower whole health index compared to individuals with fewer conditions. Individuals with common yet manageable conditions, such as diabetes, dyslipidemia, and depression, have higher whole health index compared to individuals with the more serious conditions of COPD, lung cancer, stroke, chronic kidney disease and heart failure (as shown in FIG. 10).

The whole health index represents a shift in how health can be viewed and/or measured. The whole health index can help provide a comprehensive picture of whole-person health, combining 93 measures that are representative of social, physical, and clinical factors of health, aligning with the WHO's definition of health. The whole health index is a practical, valid, and reliable tool for population health management as it provides a numeric, objective, and comprehensive measure of population health at different geographic levels and by various population segments.

The whole health index may combine multiple data sources and measure types, including publicly available data, claims data, clinical data, process and outcome measures, at individual and area levels, thereby creating a reliable measure of whole-person health that is useful not only for measuring and tracking health, but also for guiding actions to improve health both at individual and population levels.

When used to measure population health at geographic level, the whole health index has notable advantages over publicly available health indices in the US. First, because the whole health index employs individual-level data, comparisons can be made across different states, as compared to ranking counties within a given state. It also enables analyses on health disparities by population segments. Second, the whole health index can be used to track progress over time because the whole health index uses the baseline year as a benchmark to determine scores, as compared to using values from other counties or geographic units in the same years. For example, if all populations have improved health by the same amount in a given year, then the whole health index will be able to represent the improvement of health, as indicated by higher scores for the given year, compared to the baseline year; whereas other publicly available rankings that are primarily based on peer comparisons in the same year may not show any changes in their scores. This feature allows the whole health index to be used for tracking trends or improvement over time, which is not a common feature among publicly available health indices. Lastly, the whole health index has more timely data, given that many of the indicators fed into the Index were drawn from clinical, claims and enrollment data which are refreshed frequently.

The whole health index may be used to inform program planning. For example, the whole health index may be used to identify members with high social- and clinical-needs to receive a high-touch campaign to improve influenza vaccination rates. In one instance, members in the bottom 25th percentile of the whole health index in several states were contacted. These members often have multiple physical, behavioral, and social conditions that make them high risk for severe influenza symptoms; moreover, they are often harder to reach. Through this high-touch campaign, these members received additional outreach if they still were unvaccinated towards the end of the year. It is also possible to partner with community partners to ensure access to vaccines through transportation assistance and pop-up events. Preliminary results show that these high need members were vaccinated at higher rates than other members within the same insurance types. Additionally, the whole health index can be used to inform the rollout of programs to prevent obesity and improve medication adherence for Medicaid populations; these programs can be offered first in in counties with lowest whole health index scores. These examples demonstrate that the whole health index allows health plans to offer more resources and comprehensive support to those who are most in need.

The whole health index may provide a comprehensive view of whole-person health in the social context an individual lives in every day. This information may allow health plans to partner effectively across multiple care teams to co-develop solutions to address an individual's most critical needs because it provides information that may not be readily available or observable to a single care team. For example, a program can enable cross-cutting partnerships across multiple care teams to streamline touchpoints and best support members. Leveraging each domain score, care teams can quickly identify if there may be potential needs beyond their clinical program offering, and work with corresponding internal care teams and external vendors to provide additional care solutions, such as meal delivery services, transportation support, or hearing aid consultation, to improve whole health. The whole health index can be a useful tool to triage members to determine solutions for specific health and social needs.

Improving population health requires partnership and collaboration across multiple stakeholders. The whole health index and its transparent scoring method may be used as a tool allowing multiple stakeholders work together in tracking progress in health improvement and identifying targeted population to achieve common goals. The whole health index can be adopted across healthcare ecosystems. Organizations that have access to administrative claims, electronic health records, or comprehensive care history, including but not limited to, government entities, public health departments, health plans, provider organizations, and integrated health care systems, can compute the whole health index for their populations based on the techniques described herein. Payer claims data and open claims data source sourced from cleaning houses may be used to compute the whole health index. The whole health index summary results may be provided across the health care industry so that those with limited data access or resources can use summary results to help guide population health management efforts.

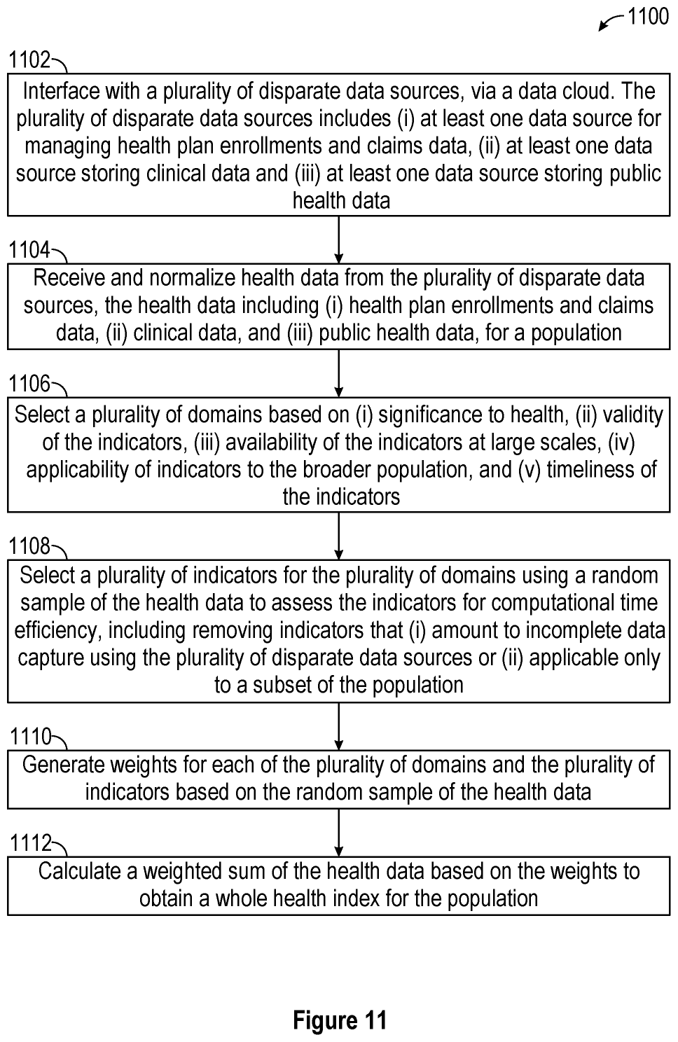

FIG. 11 shows a flowchart of an example method 1100 for calculating whole health index, according to some embodiments. The method is performed at the server 102 having one or more processors, memory, one or more displays, and one or more programs stored in the memory and configured for execution by the one or more processors, the one or more programs comprising instructions for performing the steps described herein.

The method includes interfacing (1102) (e.g., by the interface module 204) with a plurality of disparate data sources, via a data cloud (e.g., via the network 106). The plurality of disparate data sources includes (i) at least one data source for managing health plan enrollments and claims data (e.g., the health plan and claims 104), (ii) at least one data source storing clinical data (e.g., the clinical data 107), and (iii) at least one data source storing public health data (e.g., the public health data 108)

The method also includes receiving and normalizing (1104) (e.g., by the data normalizing module 208) health data (e.g., the health data 206) from the plurality of disparate data sources, the health data including (i) health plan enrollments and claims data, (ii) clinical data, and (iii) public health data, for a population (e.g., the population 112). For example, comorbidity score may range from 0-33 (using Elixhauser as an example); age may range from 0-100+, sex may be binary (Male versus Female, for example), median household income may range from $10,000 to $500,000, walkability data may range from 1 to 18.33, and affordability may range from 0% to 67%. Because data arrives in different format and ranges, some embodiments normalize the health data so as to create a composite score that is valid and reliable. For example, global health score may be converted to a ranking based on scores in a specific year (e.g., 2021), affordability may be converted to a ranking based on data for the specific year, and so on.

The method also includes selecting (1106) (e.g., by the domain selection module 212) a plurality of domains and a plurality of indicators based on (i) significance to health, (ii) validity of the indicators, (iii) availability of the indicators at large scales, (iv) applicability of indicators to the broader population, and (v) timeliness of the indicators.

The method also includes selecting (1108) (e.g., by the indicator selection module 216) a subset of indicators for the plurality of domains, including removing indicators that (i) amount to incomplete data capture using the plurality of disparate data sources or (ii) applicable only to a subset of the population. These indicators or measures are removed so that the whole health index computed is a valid measure of health across the population. Some embodiments use a random sample of the health data to assess the indicators for computational time efficiency (e.g., by the computational efficiency module 218). The random sample helps source data from multiple pipelines with the candidate measures, and helps reduce analysis time (the development dataset may be large that may take excessive time for statistical software to process for data management or statistical analyses). The computation time efficiency assessment may include measuring and/or calculating the amount of time for analyzing the data and/or the amount of processor resources that analyses requires.

The method also includes generating (1110) (e.g., by the weight generation module 220) weights (e.g., the weights 222) for each of the plurality of domains and the plurality of indicators based on the random sample of the health data. In some embodiments, generating the weights includes calculating the whole health index under a plurality of weighting schemes to determine weighting schemes across the plurality of domains and subdomains, and selecting a final weighting scheme based on an option that yields validation results in accordance with a predetermined criterion. In some embodiments, selecting the final weighting scheme includes examining the criterion validity of the whole health index by analyzing Spearmen correlation between average whole health index at a county level and public health indicators including length of life (life expectancy) and quality of life (self-reported healthy days measures, including CDC healthy days, frequent mental distress, frequent physical distress, mental health day, and/or physically healthy days. In some embodiments, selecting the final weighting scheme includes assessing if the whole health index reflects known differences in health across different populations, based on age groups, sex, race/ethnicities, rural/urban status, and/or insurance types, and selecting the final weighting scheme by determining if a scheme yields a predetermined level of performance in terms of criterion validity and discriminant validity.

The method also includes calculating (1112) (e.g., by the WHI calculation module 224) a weighted sum of the health data based on the weights to obtain a whole health index (e.g., the whole health index 226) for the population.

In some embodiments, the method further includes, in accordance with a determination that individuals in the whole population have missing scores in global health or clinical quality domains, imputing scores based on median domain score of other individuals in the same age band and sex living in the same state, respectively.

In some embodiments, the method further includes, in accordance with a determination that individuals in the whole population have missing social driver scores (primarily due to missing address information), imputing social driver score based on median value among other individuals with the same insurance types and living in the same state.

In some embodiments, generating the weights includes validating the whole health index on a predetermined portion of the health data, including analyzing Spearman correlation between average whole health index at county level and predetermined health indicators at county-level, based on health indicators comprising length of life (life expectancy) and quality of life (including CDC healthy days, frequent mental distress, frequent physical distress, mental health days, and physically healthy days.

In some embodiments, generating the weights includes assessing validity of whole health index, including estimating construct validity of composite of the whole health index, computing correlations (Pearson, Spearman, and intra-class) between three domains, conditioning these correlations on number of conditions present.

In some embodiments, generating the weights includes assessing discriminant validity by determining if the whole health index scores reflect an expected impact of clinical conditions, including assessing if individuals with multiple conditions have lower whole health index on average compared to individuals without multiple conditions, and if individuals with more severe health conditions have lower whole health index compared to those with less severe health conditions.

In some embodiments, generating the weights includes evaluating reliability of the whole health index at varying levels of geography by assessing stability of the whole health index scores.

In some embodiments, evaluating reliability includes computing split-half reliability of the whole health index scores at county and 5-digit ZIP Code levels by performing one or more of the following steps: splitting individuals within a geographical level into two groups using random sampling; computing area-level whole health index scores in both samples; and computing Pearson, Spearman, and intra-class correlations for the whole health index scores across two samples.

In some embodiments, evaluating reliability includes assessing precision of whole health index scores across various levels of geography, including computing within geographic unit variance to between geographic unit variance (WGVBGV) of the whole health index scores at census tract, 5-digit ZIP Code, and county level. WGVBGV, using the terminology of a signal-to-noise ratio, is the ratio of signal variance to the sum of the signal and noise variances (total variance in the measure). The WGVBGV statistic, ranging from 0 to 1, summarizes the proportion of the total variation in the whole health index scores at the area level due to differences between areas (considered as the signal) in relation to individual-level variation within each area (considered noise for the purposes of this test). If WGVBGV is equal to 1, variation in whole health index scores is due to differences in quality observed at the geographic level. If WGVBGV is close to zero, whole health index scores are not driven by differences in health but rather by random variation and will therefore not be useful to compare health across areas.

In some embodiments, the method further includes subdividing the health data corresponding to social drivers domain into data for six subdomains for (1) financial strain, (2) healthcare affordability, (3) food insecurity, (4) transportation barriers, (5) housing insecurity, and (6) minority status and language. The method may further include generating weights for each of the subdomains, which in turn may include calculating subdomain scores by combining individual and area-level data with equal weights, and in accordance with a determination that individuals did not have individual-level social driver data, using area-level data for the subdomain scores. The method may further include calculating the weighted domain for the social drivers domain by summing percentiles of each subdomain multiplied by a weighting factor.

In some embodiments, the method further includes subdividing the health data corresponding to clinical quality domain into data for six subdomains for (1) access to care, prevention, and screening, (2) acute care and care coordination, (3) overuse, appropriateness, and safety, (4) cardiovascular conditions, diabetes, oncology, and respiratory conditions, (5) behavioral health, and (6) women's health. The method may further include generating weights for each of the subdomains. Generating weights for the subdomains may include assigning higher weights to subdomains with more measures and more direct impact on wellbeing than other subdomains. Generating weights for the subdomains may include identifying measures within each subdomain as either a process or an outcome measures, and using a 1:3 process-to-outcome ratio to weight outcome measures more heavily. Generating weights for the subdomains may include calculating subdomain scores by combining individual and area-level data with equal weights. Generating weights for the subdomains may include scoring individuals only for measures they are qualified for. The method may further include calculating the weighted domain for the clinical quality domain by summing percentiles of each subdomain multiplied by a weighting factor.

In some embodiments, the method further includes providing each domain score to a plurality of computing resources corresponding to care teams to identify potential needs beyond their clinical program offering, and to provide additional care solutions, such as meal delivery services, transportation support, or hearing aid consultation, to improve whole health for the population.

In some embodiments, the method further includes using the whole health index to direct members of the population to appropriate solutions for their specific health and social needs.

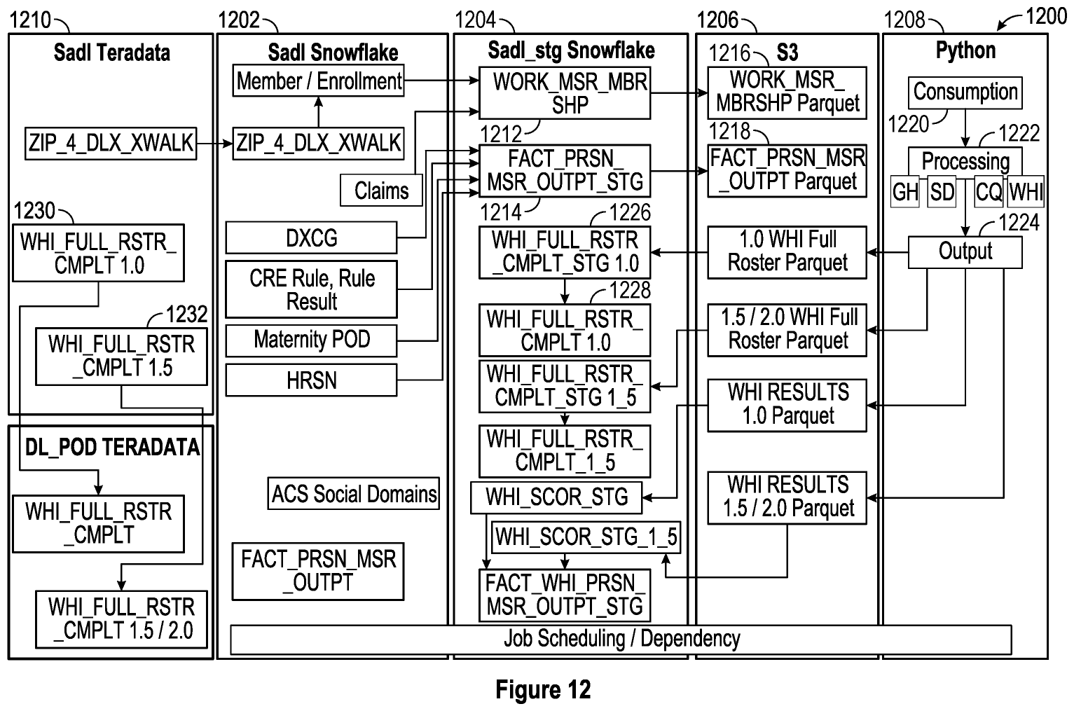

FIG. 12 is a block diagram of an example system 1200 for whole health index calculation, according to some embodiments. The system includes a cloud data warehouse 1202 (e.g., an elastically scalable cloud data warehouse, such as Snowflake) coupled to a staging module 1204. The cloud data warehouse 1202 loads data from one or more data warehouses (sometimes referred to as data sources) (e.g., optionally, via a relational database management system 1210, such as Teradata). Data loads, reloads and/or updates may be performed on a predetermined schedule, and/or based on monitoring the data sources for updates. This data is initially loaded into one or more tables in the cloud data warehouse 1202. The data from the one or more tables is extracted, transformed and loaded (ETL) into tables 1212 and 1214 in the staging module 1204, using one or more ETL scripts (e.g., using ExecuteSql Glue job (ANTM-EDL-prod-Gj-ExecuteSql)). Protegrity detok functions may be handled in the ETL scripts as required. The cloud data warehouse 1202 is the user-facing data at the transactional level, which may include enrollment claims. The staging module 1204 is the development environment and may have files summarized specifically for submission to Python and files received back from the Python process. The table 1212 is one row per member with demographic information. The table 1214 includes many rows per member as it contains the measures from clinical quality (CQ), social drivers (SD), and global health (GH) domains. The table 1214 may contain up 75 or more measures for a member. Next, the data in the tables 1212 and 1214 are unloaded into corresponding tables 1216 and 1218 in an object storage service 1206 (e.g., a cloud service for storage, such as S3); ad-hoc unload jobs may be used (e.g., Glue job (ANTM-EDL-prod-Gj-AdhocUnloadJob)). The object storage service 1206 support handoffs in file format between Python process 1208 and the cloud data warehouse 1202. Subsequently, the data in the tables 1216 and 1218 are unloaded in the form of parquet files to Python process 1208 (any language that is similar to Python may be used for this purpose). The Python process 1208 reads (1220) the unloaded data, processes (1222) the data and generates (1224) output files to the object storage service 1206. The output files generated on the object storage service 1206 are loaded into tables 1226 and 1228 using an ingestion framework. The ingestion framework may be invoked after the files are available in the source path. The data in the tables 1226 and 1228 are subsequently copied into final tables 1230 and 1232, respectively, in the relational database management system 1210, using ETL scripts.

In some embodiments, the cloud data warehouse 1202 publishes a member record for each person. Towards that, the cloud data warehouse 1202 may gather and/or determines a “best” member demographics across multiple member enrollment records during an experience period, and/or gather member claims across all enrollments for claims incurred during experience period. In some embodiments, the cloud data warehouse 1202 publishes a set of measures for each person. Towards that, the cloud data warehouse 1202 may gather global health or age, sex, and comorbidity scores information for a member during the experience period, gather clinical quality data (e.g., data for 60 measures) for the member during the experience period, gather maternity clinical measures for the member during the experience period, gather social needs information from member level assessment or claims data that note health related social needs (HRSN), and/or consume publicly available American community survey (ACS) data and derive SVI data for each FIPS code.

In some embodiments, the Python process 1208 receives member and measure files, establishes defaults for member data, processes measure data; converting from row based to column based file, processes global health measure (e.g., establish population-based default when global health value not available, and/or derive global health score), processes clinical quality measures (establish population-based default when CG value not available, derive clinical quality score), process social driver measures (e.g., establish population-based default when SD value is not available, derive domain based scores, derive social driver score), derive WHI Score, and/or publish file with domain-specific scores to populate to the cloud data warehouse 1202. The cloud data warehouse 1202 receives GH, CQ, SQ, WHI scores and publishes for enterprise consumption.

While embodiments and alternatives have been disclosed and discussed, the invention herein is not limited to the particular disclosed embodiments or alternatives but encompasses the full breadth and scope of the invention including equivalents, and the invention is not limited except as set forth in and encompassed by the full breadth and scope of the claims herein.

What is claimed is:

1. A system for calculating whole health index, the system comprising:
an interface module comprising a cloud-native platform configured to:
interface with a plurality of disparate data sources, via a data cloud, wherein the plurality of disparate data sources includes (i) at least one data source for managing health plan enrollments and claims data, (ii) at least one data source for storing clinical data, and (iii) at least one data source storing public health data;
monitor the plurality of data sources for updates to health data and/or at predetermined time intervals to identify health receipt time periods; and
receive the health data from the plurality of data sources, according to the health receipt time periods, via a database management system, including extracting, transforming and loading the health data into one or more tables, wherein the health data includes (i) health plan enrollments and claims data, (ii) clinical data, and (iii) public health data, for a population;
a data normalizing module coupled to the interface module, configured to:
obtain the health data from the one or more tables; and
normalize the health data from the plurality of data sources including reformatting and/or range fitting comorbidity scores, age ranges, median household income ranges, walkability data ranges, and affordability ranges;
a domain selection and indicator selection module coupled to the data normalizing module, configured to:
select a plurality of domains and a plurality of indicators based on (i) significance to health, (ii) validity of the indicators, (iii) availability of the indicators at large scales, (iv) applicability of indicators to the population, and (v) timeliness of the indicators; and
select a subset of indicators for the plurality of domains including removing indicators that (i) amount to incomplete data capture using the plurality of disparate data sources or (ii) applicable only to a subset of the population;
a weight generation module coupled to the domain selection and indicator selection module, configured to:
generate weights for each of the plurality of domains and the subset of indicators based on a random sample of the health data; and
a whole health index calculation module coupled to the weight generation module, configured to:
calculate a weighted sum of the health data based on the weights to obtain a whole health index for the population.

2. The system of claim 1, wherein the weight generation module is configured to:
calculate the whole health index under a plurality of weighting schemes to determine weighting schemes across the plurality of domains and subdomains; and
select a final weighting scheme based on an option that yields validation results in accordance with a predetermined criterion.

3. The system of claim 2, wherein the weight generation module is configured to select the final weighting scheme by examining the predetermined criterion validity of the whole health index by analyzing Spearmen correlation between average whole health index at a county level and public health indicators including length of life and quality of life.

4. The system of claim 3, wherein the weight generation module is configured to select the final weighting scheme by (i) assessing if the whole health index reflects known differences in health across different populations, based on age groups, sex, race/ethnicities, rural/urban status, and/or insurance types, and (ii) selecting the final weighting scheme by determining if a scheme yields a predetermined level of performance in terms of criterion validity and discriminant validity.

5. The system of claim 1, wherein the whole health index calculation module is further configured to:
in accordance with a determination that individuals in the population have missing scores in global health or clinical quality domains, impute scores based on median domain score of other individuals in a same age band and sex living in a same state, respectively.

6. The system of claim 1, wherein the whole health index calculation module is further configured to:
in accordance with a determination that individuals in the population have missing social driver scores, impute social driver score based on median value among other individuals with same insurance types and living in a same state.

7. The system of claim 1, wherein the weight generation module is configured to:
validate the whole health index on a predetermined portion of the health data, including analyzing Spearman correlation between average whole health index at county level and predetermined health indicators at county-level, based on health indicators comprising length of life and quality of life.

8. The system of claim 1, wherein the weight generation module is configured to:
assess validity of whole health index, including estimating construct validity of composite of the whole health index, computing correlations between three domains, conditioning these correlations on number of conditions present.

9. The system of claim 1, wherein the weight generation module is configured to:
assessing discriminant validity by determining if the whole health index reflects an expected impact of clinical conditions, including assessing if individuals with multiple conditions have lower whole health index on average compared to individuals without multiple conditions, and if individuals with more severe health conditions have lower whole health index compared to those with less severe health conditions.

10. The system of claim 1, wherein the whole health index calculation module is further configured to:
evaluate reliability of the whole health index at varying levels of geography by assessing stability of the whole health index.

11. The system of claim 10, wherein evaluating reliability comprises:
computing split-half reliability of the whole health index at county and 5-digit ZIP code levels by:
splitting individuals within a geographical level into two groups using random sampling;
computing area-level whole health index scores in both samples; and
computing Pearson, Spearman, and intra-class correlations for the whole health index across two samples.

12. The system of claim 10, wherein evaluating reliability comprises:
assessing precision of whole health index scores across various levels of geography, including computing within geographic unit variance to between geographic unit variance (WGVBGV) of the whole health index scores at census tract, 5-digit ZIP Code, and county level, wherein WGVBGV is a ratio of signal variance to a sum of signal and noise variances, wherein the WGVBGV statistic, ranging from 0 to 1, summarizes proportion of total variation in the whole health index scores at an area level due to differences between areas in relation to individual-level variation within each area, wherein if WGVBGV is equal to 1, variation in whole health index scores is due to differences in quality observed at a geographic level, and wherein if WGVBGV is close to zero, whole health index scores are not driven by differences in health but rather due to random variation and will therefore not be useful to compare health across areas.

13. The system of claim 1, wherein the domain selection and indicator selection module is further configured to:
subdivide the health data corresponding to social drivers domain into data for six subdomains for (1) financial strain, (2) healthcare affordability, (3) food insecurity, (4) transportation barriers, (5) housing insecurity, and (6) minority status and language;
wherein the weight generation module is further configured to:
generate weights for each of the subdomains, including:
calculating subdomain scores by combining individual and area-level data with equal weights; and
in accordance with a determination that individuals did not have individual-level social driver data, using area-level data for the subdomain scores; and
calculate the weighted sum for a social drivers domain by summing percentiles of each subdomain multiplied by a weighting factor.

14. The system of claim 1, wherein the domain selection and indicator selection module is further configured to:
subdivide the health data corresponding to clinical quality domain into data for six subdomains for (1) access to care, prevention, and screening, (2) acute care and care coordination, (3) overuse, appropriateness, and safety, (4) cardiovascular conditions, diabetes, oncology, and respiratory conditions, (5) behavioral health, and (6) women's health;
wherein the weight generation module is further configured to:
generate weights for each of the subdomains, including:
assigning higher weights to subdomains with more measures and more direct impact on wellbeing than other subdomains;
identifying measures within each subdomain as either a process or an outcome measures, and using a 1:3 process-to-outcome ratio to weight outcome measures more heavily;
calculating subdomain scores by combining individual and area-level data with equal weights; and
scoring individuals only for measures they are qualified for; and
calculate the weighted sum for a clinical quality domain by summing percentiles of each subdomain multiplied by a weighting factor.

15. The system of claim 1, wherein the whole health index calculation module is further configured to:
provide each domain score to a plurality of computing resources corresponding to care teams to identify potential needs beyond their clinical program offering, and to provide additional care solutions, such as meal delivery services, transportation support, or hearing aid consultation, to improve whole health for the population.

16. The system of claim 1, wherein the whole health index calculation module is further configured to:
use the whole health index to direct members of the population to appropriate solutions for their specific health and social needs.

17. A method of calculating whole health index, performed at a computer system having one or more processors, memory, one or more displays, and one or more programs stored in the memory and configured for execution by the one or more processors, the one or more programs comprising instructions for:
interfacing with a plurality of disparate data sources, via a data cloud, wherein the plurality of disparate data sources includes (i) at least one data source for managing health plan enrollments and claims data, (ii) at least one data source storing clinical data, and (iii) at least one data source storing public health data;
receiving and normalizing health data from the plurality of disparate data sources, the health data including (i) health plan enrollments and claims data, (ii) clinical data, and (iii) public health data, for a population;
selecting a plurality of domains and a plurality of indicators based on (i) significance to health, (ii) validity of the indicators, (iii) availability of the indicators at large scales, (iv) applicability of indicators to the population, and (v) timeliness of the indicators;
selecting a subset of indicators for the plurality of domains including removing indicators that (i) amount to incomplete data capture using the plurality of disparate data sources or (ii) applicable only to a subset of the population;
generating weights for each of the plurality of domains and the subset of indicators based on a random sample of the health data; and
calculating a weighted sum of the health data based on the weights to obtain a whole health index for the population.

18. The method of claim 17, wherein generating the weights comprises:
calculating the whole health index under a plurality of weighting schemes to determine weighting schemes across the plurality of domains and subdomains; and
selecting a final weighting scheme based on an option that yields validation results in accordance with a predetermined criterion.

19. The method of claim 18, wherein selecting the final weighting scheme comprises:
examining the predetermined criterion validity of the whole health index by analyzing Spearmen correlation between average whole health index at a county level and public health indicators including length of life and quality of life.

20. A non-transitory computer readable storage medium storing one or more programs configured for execution by a computer system having a display, memory and one or more processors, the one or more programs comprising instructions for:
interfacing with a plurality of disparate data sources, via a data cloud, wherein the plurality of disparate data sources includes (i) at least one data source for managing health plan enrollments and claims data, (ii) clinical data, and (iii) at least one data source storing public health data;
receiving and normalizing health data from the plurality of disparate data sources, the health data including (i) health plan enrollments and claims data, (ii) clinical data and (iii) public health data, for a population;
selecting a plurality of domains and a plurality of indicators based on (i) significance to health, (ii) validity of the indicators, (iii) availability of the indicators at large scales, (iv) applicability of indicators to the population, and (v) timeliness of the indicators;
selecting a subset of indicators for the plurality of domains including removing indicators that (i) amount to incomplete data capture using the plurality of disparate data sources or (ii) applicable only to a subset of the population;
generating weights for each of the plurality of domains and the subset of indicators based on a random sample of the health data; and
calculating a weighted sum of the health data based on the weights to obtain a whole health index for the population.
